(19) United States
(12) Patent Application Publication
(10) Pub. No.: US 2008/0249389 A1
(43) Pub. Date: Oct. 9, 2008

(54) Electrode lead set for measuring physiologic information

(75) Inventors: Brian Erik Haug, Portland, OR (US); James Francis McIntire, West Linn, OR (US)

Correspondence Address: Marguerite E. Gerstner;Tyco Electronics Corporation, 307 Constitution Drive, M/S R20/2B, Menlo Park, CA, 94025-1164, US

(73) Assignee: Tyco Electronics Corporation

(21) Appl. No.: 11/732,372

(22) Filed: Apr. 3, 2007

(51) Int. Cl. A61N 1/05

(52) U.S. Cl. 600/372

(57) ABSTRACT

An electrode lead set is provided for electrical connection to a body. The electrode lead set includes a flexible substrate core extending between a proximal end portion and a distal end portion. The distal end portion includes a plurality of branch end portions that are each configured to hold an electrode. The substrate core is separable into a plurality of branches that are each joined to adjacent branches by a separable interface prior to separation. Each of the plurality of branches includes a corresponding one of the branch end portions. Prior to separation, the plurality of branches are configured such that the branch end portions are maintained in an array at the distal end portion of the substrate core. An electrically conductive pathway extends along each branch from the corresponding branch end portion toward the proximal end portion of the substrate core.

### BACKGROUND OF THE INVENTION

The invention relates generally to electrode lead sets, and, more particularly, to electrode lead sets for measuring physiologic information.

An electrocardiograph (ECG) system monitors heart electrical activity in a patient. Conventional ECG systems utilize electrodes placed on a patient in specific locations to detect electrical impulses generated by the heart during each beat. Typically, the electrical impulses or signals are detected by and directly transferred from the electrodes to a stationary ECG monitor via multiple cables or wires. The ECG monitor performs various signal processing and computational operations to convert the raw electrical signals into meaningful information that can be displayed on a monitor or printed out for review by a physician.

ECG measurements are taken by applying electrodes to different chest locations and additional body locations, such as the arms and legs. In the past, each of the electrodes has been connected to the ECG monitor by a separate shielded lead. However, the separate leads sometimes become entangled with each other during use and/or during application of the electrodes to the various body locations. Entanglement of the leads may make it more difficult and/or time-consuming to apply the electrodes, which may delay diagnosis and/or increase the time, and therefore cost, of the ECG procedure, as well as possibly inconveniencing the patient. Entanglement of the leads may be a minor inconvenience during routine medical procedures, such as an annual check-up. However, entanglement may be life threatening during emergency situations in which an immediate ECG read-out is critical.

To reduce entanglement of the electrode leads, some known ECG systems embed the electrodes and the corresponding leads within an insulative sheet of material that is placed over the patient's chest area and/or the other additional body locations. The electrodes are embedded within the sheet at fixed locations that, when the insulative sheet is placed over the patient's body, correspond to the desired locations on the patient for taking ECG measurements. However, because body size and/or shape may vary greatly between different patients, the fixed location of one or more of the electrodes within the insulative sheet may not correspond to the desired location for taking ECG measurements on some patients. For example, the locations of electrodes within an insulative sheet designed for a man over six feet tall may not align with the desired locations for taking ECG measurements on the body of a woman who is about five feet tall. Accordingly, different insulative sheets may be designed for different body sizes and/or shapes, which may increase a cost of the insulative sheets as well as a cost of performing the ECG procedure.

Moreover, the leads of some known ECG systems are typically reused many times on a number of different patients over the field life of the ECG lead set. To prevent the transmission of infection between patients, the electrodes and leads are disinfected between uses. However, the disinfection process may add time and/or expense to the ECG procedure. Moreover, the disinfection process sometimes fails to completely disinfect the electrodes and/or leads, for example because of human or machine error. In some cases, the disinfection process may be neglected completely. As a result, patient-to-patient infection caused by the reuse of ECG electrode lead sets has become an area of concern among healthcare providers. In addition to the general concern for the patient's well being, settlements and/or law suits resulting from patient-to-patient infection can be costly for healthcare providers.

There is a need for an electrode lead set having leads that are less likely to be entangled, that accommodate differently sized and/or shaped patient bodies, and/or that facilitate reducing patient-to-patient infection.

### BRIEF DESCRIPTION OF THE INVENTION

In one embodiment, an electrode lead set is provided for electrical connection to a body. The electrode lead set includes a flexible substrate core extending between a proximal end portion and a distal end portion. The distal end portion includes a plurality of branch end portions that are each configured to hold an electrode. The substrate core is separable into a plurality of branches that are each joined to adjacent branches by a separable interface prior to separation. Each of the plurality of branches includes a corresponding one of the branch end portions. Prior to separation, the plurality of branches are configured such that the branch end portions are maintained in an array at the distal end portion of the substrate core. An electrically conductive pathway extends along each branch from the corresponding branch end portion toward the proximal end portion of the substrate core.

In another embodiment, an electrode lead set assembly is provided for electrical connection to a body. The electrode lead set assembly includes a flexible substrate core extending between a proximal end portion and a distal end portion. The distal end portion includes a plurality of branch end portions. The substrate core is separable into a plurality of branches that are each joined to adjacent branches by a separable interface prior to separation. Each of the plurality of branches includes a corresponding one of the branch end portions. Prior to separation, the plurality of branches are configured such that the branch end portions are maintained in an array at the distal end portion of the substrate core. A plurality of electrodes are each held by a corresponding branch end portion of a different branch of the plurality of branches. A plurality of electrically conductive pathways each extend along a different branch of the plurality of branches. Each electrically conductive pathway is electrically connected to the corresponding electrode held by the corresponding branch. Each electrically conductive pathway extends from the corresponding branch end portion toward the proximal end portion of the substrate core.

### BRIEF DESCRIPTION OF THE DRAWINGS

FIG. 1 is a top plan view of an exemplary embodiment of an electrode lead set for electrical connection to a body.

FIG. 2 is a top plan view of an exemplary alternative embodiment of an electrode lead set.

FIG. 3 is a top plan view of another exemplary alternative embodiment of an electrode lead set.

FIG. 4 is a top plan view of a portion of the electrode lead set shown in FIG. 1 illustrating an exemplary embodiment of a separable interface between branches of the electrode lead set.

FIG. 5 is a perspective view of the electrode lead set shown in FIG. 1 illustrating the branches of the electrode lead set being separated.

FIG. 6 is a cross-sectional view of an alternative embodiment of a branch of an electrode lead set.

FIG. 7 illustrates one exemplary embodiment of a method of using the electrode lead set embodiments described and illustrated herein.

FIG. 8 is a schematic diagram of an exemplary embodiment of an electrocardiogram (ECG) system that the electrode lead set embodiments described and illustrated herein may be used with.

FIG. 9 is a schematic diagram of an alternative exemplary embodiment of an electrocardiogram (ECG) system that the electrode lead set embodiments described and illustrated herein may be used with.

### DETAILED DESCRIPTION OF THE INVENTION

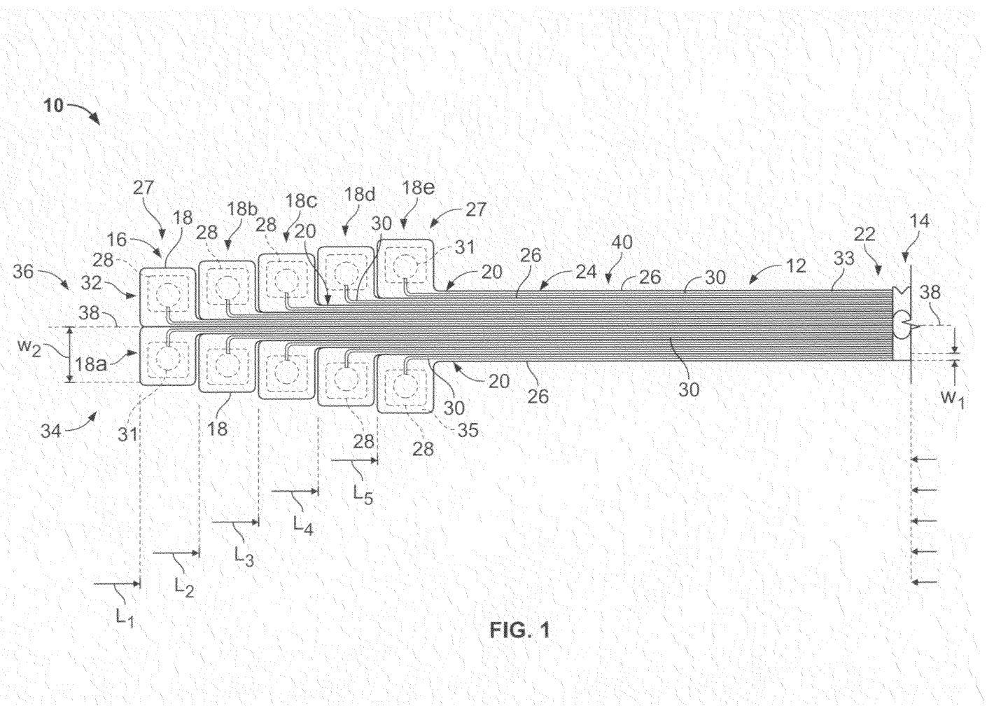

FIG. 1 is a plan view of an exemplary embodiment of an electrode lead set 10. The electrode lead set 10 includes a flexible substrate core 12 that extends along a length L₁ from a proximal end portion 14 to a distal end portion 16. The distal end portion 16 includes a plurality of pads 18 that are each integrally formed as a portion of the substrate core 12. The substrate core 12 is separable along a portion of the length of the substrate core 12 to define a plurality of branches 20. Specifically, the substrate core 12 includes a base portion 22 that includes the proximal end portion 14 and a branch portion 24 extending from the base portion 22 to the distal end portion 16. The substrate core 12 is separable along a separable interface 25 (FIG. 4) extending along at least a portion of the length of the branch portion 24 into the plurality of branches 20. Each branch 20 includes the corresponding pad 18 and a stem 26 that extends from the base portion 22 to a branch end portion 27 that includes the corresponding pad 18.

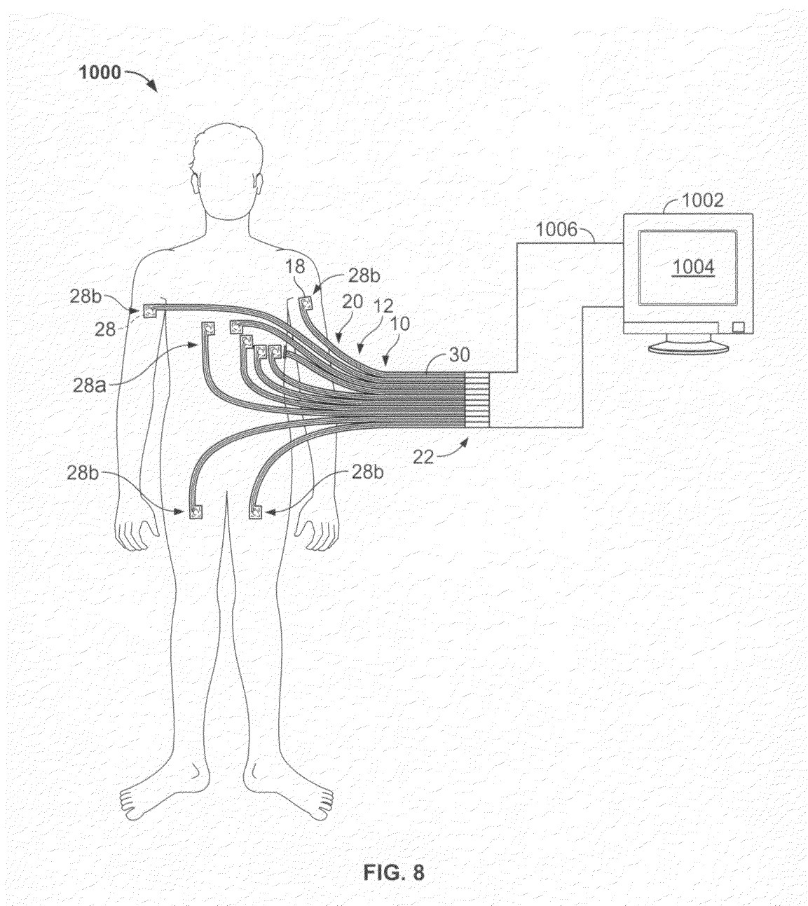

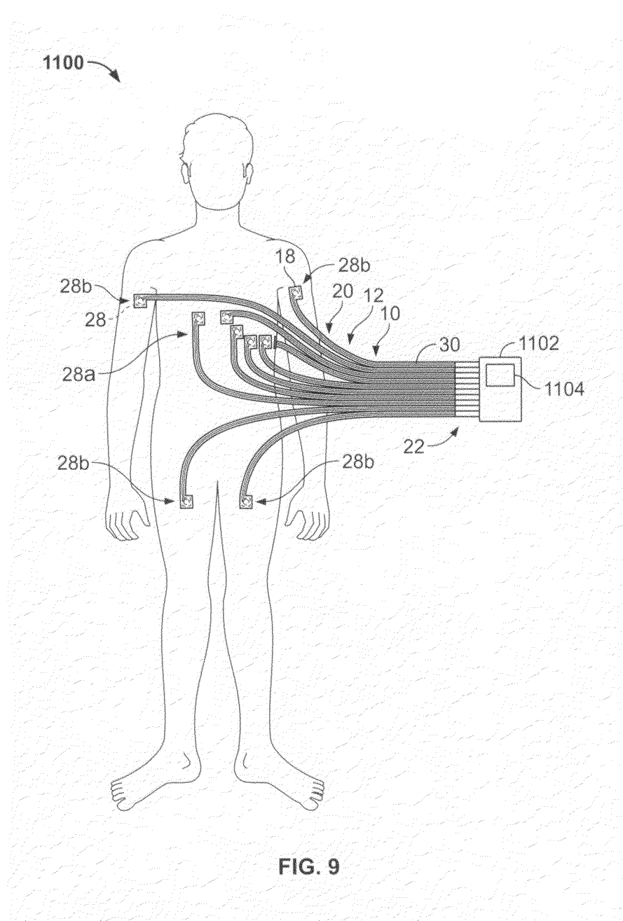

Each branch 20 is configured to hold an electrode 28. In the exemplary embodiment, each of the pads 18 holds an electrode 28. Although shown as generally rectangular, the pads 18 may have any shape. Alternatively, one or more branches 20 do not include a pad 18. As will be described in more detail below, the electrodes 28 are configured to be placed at different locations on the body for measuring physiological information of the body. The substrate core 12 includes a plurality of electrically conductive pathways 30 that extend along the length of the substrate core 12 between the proximal and distal end portions 14 and 16, respectively. In the exemplary embodiment, each electrically conductive pathway 30 extends along the length of a different stem 26 and along at least a portion of the length of the base portion 22. Alternatively, one or more of the electrically conductive pathways 30 does not extend along a portion of the base portion 22. Each of the electrically conductive pathways 30 is electrically connected to the corresponding electrode 28 to provide an electrical connection between the electrode 28 and a monitoring or other electronic device (e.g., the electrocardiogram (ECG) monitoring device 1002, shown in FIG. 8, and/or the hand-held patient monitor 1102, shown in FIG. 9) that may be connected to the base portion 22 at the proximal end portion 14.

Prior to separation of the substrate core 12 into the plurality of branches 20, the pads 18 are held in an array 32 such that pairs of the pads 18 are maintained in a uniform pattern on opposite sides 34 and 36 of a central longitudinal axis 38 of the substrate core 12. As used herein, the term “array” may include an ordered grouping of the pads 18 and/or may include a collection of the pads 18 that may be random, ordered, or a combination of random and ordered. For example, the pad array 32 includes an outermost (relative to the base portion 22) pair of pads 18a, an innermost pair of pads 18e, and a plurality of intermediate pairs of pads 18b, 18c, and 18d held between the outermost and innermost pairs of pads 18a and 18e, respectively, in an initial fixed relation to the longitudinal axis 38. By way of example, the pairs of pads 18a-e are arranged in a tiered configuration where the outermost pair of pads 18a is located a distance, or the length L₁, measured from the proximal end portion 14 of the substrate core 12. Each successive pair of pads 18b-e is located a shorter distance from the proximal end portion 14, denoted by a respective length L₂, L₃, L₄, and L₅ that is progressively shorter. The stems 26 connected to the pair of pads 18a extend along a central portion 40 of the substrate core 12 between the stems 26 connected to each of the other pairs of pads 18b-e. The stems 26 connected to each successive pair of pads 18b-e within the tiered array 32 extend directly about opposite sides of the stems 26 connected to any directly previous pair of pads within the tiered array 32. In other words, the stems 26 connected to each pair of pads 18a-e extend directly between the stems 26 connected to any directly subsequent pair of pads within the tiered array 32. In the exemplary tiered arrangement, the outermost pair of pads 18a are spaced immediately adjacent and on opposite sides of the longitudinal axis 38. Each pair of pads 18a-e is located transversely from one another by proportionally increasing distances forming a diverging tapered pattern. In other words, the intermediate and innermost pair of pads 18b-e, respectively, are located transversely from one another by different amounts to form a “v” shape.

Because each pad 18 of the pairs 18a-e extends about the same length from the proximal end portion 14 of the substrate core 12 as the other pad 18 within the same pair, the array 32 of pads 18 is symmetrical about the central longitudinal axis 38. However, the array 32 of pads 18 need not be symmetrical about the central longitudinal axis 38 to be uniform. Rather, the location of each pad 18 within a pair may be staggered relative to each other such that a uniform pattern is formed on opposite sides of the central longitudinal axis 38 that is not symmetrical about the axis 38. Moreover, the patterns illustrated herein are meant as exemplary only. The pads 18 are not limited to uniform patterns, tiered patterns, symmetrical patterns, the specific pattern of the array 32 shown in FIG. 1, or any other exemplary patterns described and/or illustrated herein. Rather, the pads 18 may have any suitable pattern that enables the electrode lead set 10 to function as described herein.

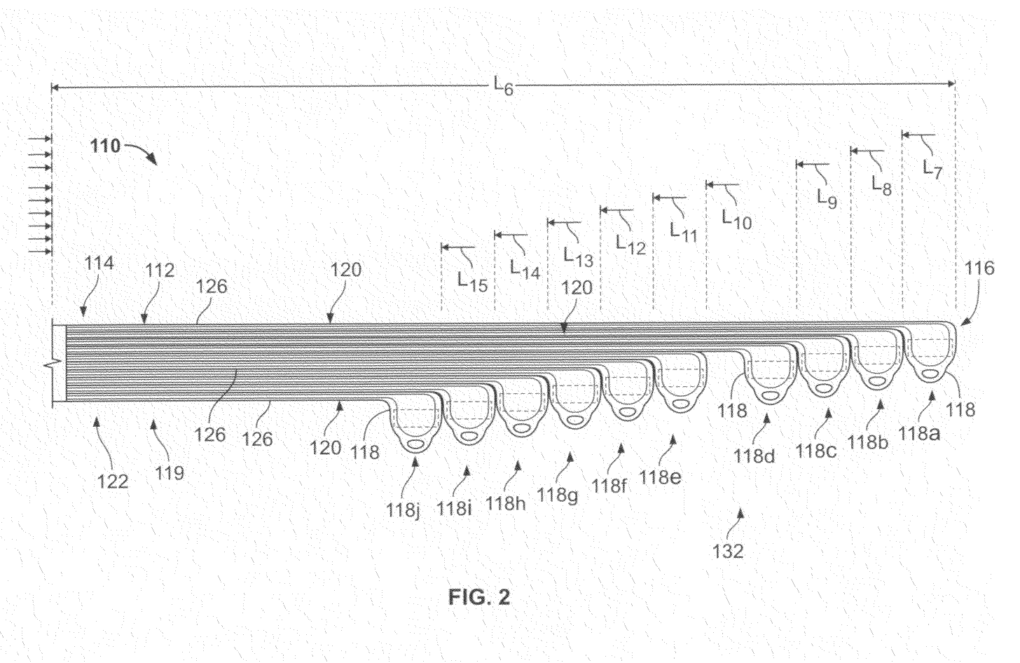

FIG. 2 illustrates an exemplary alternative embodiment of an electrode lead set 110. Prior to separation of a substrate core 112 into a plurality of branches 120, a plurality of pads 118 are held in a tiered array 132 that includes an outermost (relative to a base portion 122) pad 118a, an innermost pad 118j, and a plurality of intermediate pads 118b, 118c, 118d, 118e, 118f, 118g, 118h, and 118i held between the outermost and innermost pads 118a and 118j, respectively. The pads 118a-j are arranged in a tiered configuration where the outermost pad 118a is spaced a distance, or length L₆, measured from a proximal end portion 114 of the substrate core 112. Each successive pad 118b-j is located a shorter distance from the proximal end portion 114, denoted by a respective length L₇, L₈, L₉, L₁₀, L₁₁, L₁₂, L₁₃, L₁₄, and L₁₅ that is progressively shorter. A stem 126 connected to the pad 118j forms a portion of a side 119 of the substrate core 112. The stem 126 connected to each successive pad 118i-a (going in a direction toward a distal end portion 116 of the substrate core 112) within the tiered array 132 at least partially surrounds the stem 126 connected to any directly previous pad 118 within the tiered array 132.

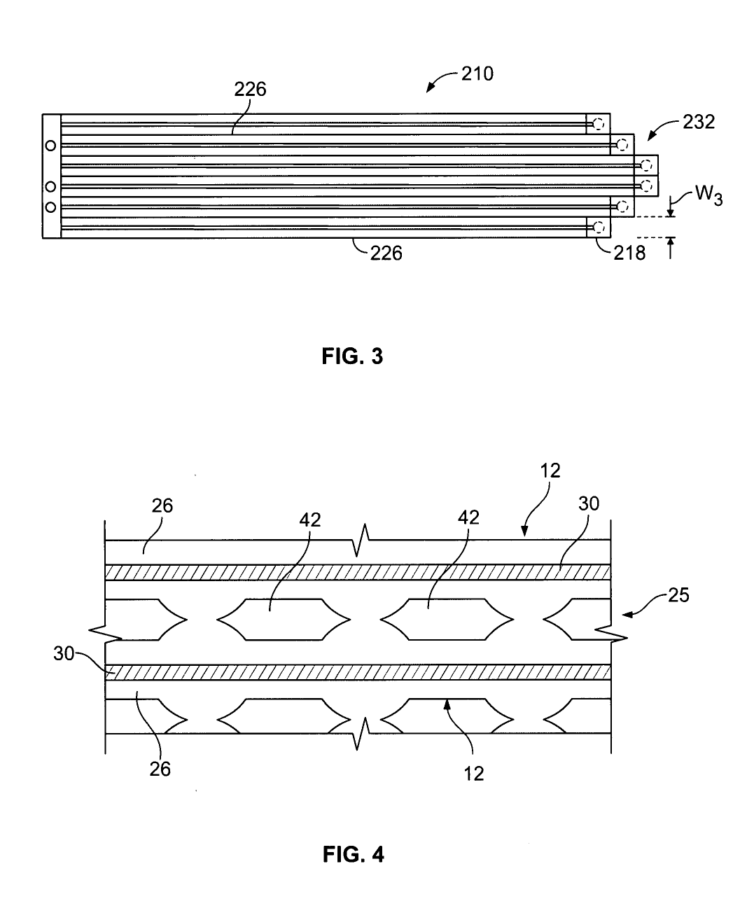

Referring again to FIG. 1, although shown in FIG. 1 as having a width W₁ that is smaller than a width W₂ of the pads 18, the stems 26 may alternatively have a width that is greater or about equal to the width of the pads 18. For example, FIG. 3 illustrates an alternative embodiment of an electrode lead set 210 having an array 232 of a plurality of pads 218 similar to the array 32 of FIG. 1 except wherein a width W₃ of each of a plurality of stems 226 is about equal to the width of each of the pads 218.

Referring again to FIG. 1, as discussed above, the branches 20 of the substrate core 12 are joined along at least a portion of a length thereof to adjacent branches 20 by a separable interface 25 (FIG. 4). Although shown as being joined along the stems 26, in addition or alternative to the separable interface 25 along the stems 26, each branch 20 may be separably joined to each adjacent branch 20 at any portion thereof, such as, but not limited to, at the pads 18. Moreover, the separable interface 25 may include any suitable configuration, arrangement, structure, means, and/or the like that enables the substrate core 12 to be separable into the plurality of branches. For example, FIG. 4 illustrates the separable interface 25 of the exemplary embodiment wherein perforations 42 are provided along at least a portion of the length of each of the stems 26. Adjacent stems 26 can be separated by breaking the connections extending between each perforation 42. The perforations 42 may have any suitable size, shape, spacing, and/or frequency in addition or alternative to how the perforations 42 are illustrated in FIG. 4. The separable interface 25 of the exemplary embodiment is meant as exemplary only. Connection between adjacent branches 20 is not limited to the perforations 42, but rather the branches 20 may be connected using any suitable structure and/or means that enables separable connection between adjacent branches 20. For example, adjacent branches 20 may additionally or alternatively be connected together using a perforated tape. Moreover, the stems 26 may be connected at any number and location(s) along their length.

The exemplary arrangements shown in FIGS. 1-3 allow the pads, and therefore the electrodes, to be nested together as shown in the respective arrays 32, 132, and 232. The nested arrangement may facilitate reducing manufacturing costs by reducing an amount of material used to fabricate a plurality of the electrode lead sets. As discussed above, the arrangements shown in FIG. 1-3 are meant as exemplary only. The branches and the corresponding pads and electrodes may have any other suitable relative arrangement that enables the electrode lead set to function as described herein. For example, the nested arrangement may be selected to facilitate placement of the electrodes at the desired locations on the body and/or to facilitate reducing manufacturing time, complexity, difficulty, and/or cost.

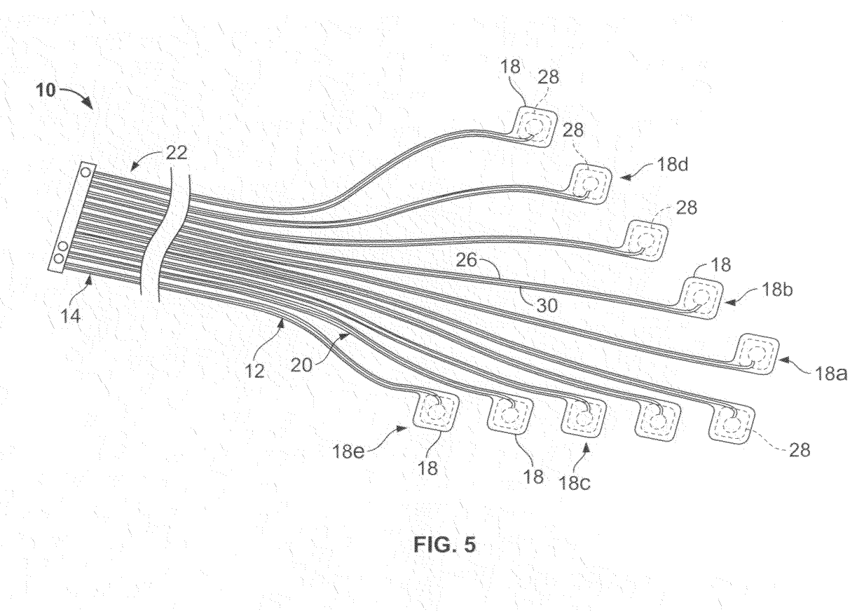

In operation, and referring to FIGS. 1 and 5, the base portion 22 of the substrate core 12 is placed on or adjacent a patient's body (not shown in FIG. 1 or 5). Before the electrodes 28 are placed on the patient's body, the branches 20 are joined to each adjacent branch 20 by the separable interface 25 (FIG. 4) such that the electrodes 28 are initially nested together in the array 32, as shown in FIG. 1. Each branch 20 can be separated from adjacent branches 20 by breaking the separable interface 25 between the branches 20. Once separated, a branch 20 can be manipulated to place the corresponding electrode 28 at the desired location on the patient's body. Because the branches 20 are initially nested together in the array 32, and each branch 20 can be separately “peeled-away” from the other branches 20, the stems 26 of the branches 20 may be less likely to entangle during placement of the electrodes 28. Although the branches 20 may be less likely to entangle, some of the branches 20 may overlap when the electrodes 28 are placed at the desired locations. In some embodiments, the pads 18 and/or the stems 26 include indicia (not shown) that indicates the desired location on the patient's body of the corresponding electrode 28.

FIG. 5 illustrates the electrode lead set 10 wherein the separable interface 25 (FIG. 4) between each pair of adjacent branches 20 has been broken. Once a branch 20 has been separated from the other branches 20, the branch 20 is only connected to the other branches 20 by the base portion 22. Because the substrate core 12 is generally flexible, and the branches 20 are only connected to each other by the base 22 (after separation), the stems 26 can bend along their length such that each pad 18, and therefore each electrode 28, is selectively positionable at a plurality of different positions relative to the other electrodes 28. Specifically, the relative arrangement of the electrodes 28 is selectable such that each electrode 28 can be independently positioned without generally affecting the position of the other electrodes 28. Accordingly, the electrodes 18 may be positionable in a variety of relative arrangements to facilitate accommodating different patient body sizes and/or shapes. For example, electrodes 28 placed on the arm and leg of a patient may have a position relative to one another that is different than when the electrodes 28 are placed on the same portions of the arm and leg of another patient. The selective relative positioning of each of the electrodes 28 may allow for greater flexibility in using the electrode set 10 with different patients.

The branches 20 may have any suitable configuration, arrangement, pattern, and/or the like (whether when joined by the separable interfaces 25, shown in FIG. 4, or when separated) that enable the electrodes 28 to be placed at the desired locations on the body. For example, the electrode lead set 10 may include any number of branches 20 for positioning any number of electrodes 28 at any number of locations on the body. In the exemplary embodiment of FIGS. 1 and 5, the electrode lead set 10 includes ten branches 20 for use within an ECG system (e.g., the ECG system 1000 shown in FIG. 8). For example, the electrodes 28 of six of the branches 20 may be configured to be placed at six different locations on a chest region of the body (e.g., the prescribed ECG precordial locations V₁, V₂, V₃, V₄, V₅, and V₆ of the American Heart Association (AHA) or the prescribed ECG precordial locations C₁, C₂, C₃, C₄, C₅, and C₆ of the International Electrotechnical Commission (IEC)) and the electrodes 28 of four branches 20 are configured to be placed on different limbs of the body (e.g., the prescribed locations RA, LA, RL, and LL of the AHA or the prescribed locations R, L, N, and F of the IEC). An example of an alternative embodiment of the electrode lead set 10 includes only three branches 20 for use within an ECG system, wherein the electrodes of the three branches 20 are configured to be placed at different limbs of the body (e.g., both arms and the left leg). Yet another example of an alternative embodiment of the electrode lead set 10 includes only five branches 20 for use within an ECG system, wherein the electrodes of the four of the five branches 20 are configured to be placed at different limbs of the body (e.g., both arms and both legs) and one of the five branches 20 is configured to be placed on the chest region of the body. Even another example of an alternative embodiment of the electrode lead set 10 includes twelve branches 20 for use within an ECG system, wherein some of the electrodes 28 of the twelve branches 20 are configured to be placed at different limbs of the body and some of the electrodes 28 of the twelve branches 20 are configured to be placed on the chest region of the body. The electrode lead set embodiments described and/or illustrated herein are not limited to the three, five, ten, and twelve branch embodiments described and/or illustrated herein, but rather may include any number of branches for positioning any number of electrodes at any number of locations on the body.

The stems 26 of each of the branches 20 may have any suitable length that enables the corresponding electrode 28 to be placed at the corresponding desired location on the body. For example, in the exemplary embodiment of FIGS. 1 and 5 the stems 26 are each between about 15 inches (38.1 centimeters) and about 48 inches (121.9 centimeters) long. Another example is each stem 26 being between about 20 inches (50.8 centimeters) and about 32 inches (81.3 centimeters) long. The relative length of each of the stems 26, the pattern of any arrays of the pads 18 and/or electrodes 28, and/or the particular location of each of the pads 18 and/or electrodes 28 within the array and relative to each other (whether when joined by the separable interface 25 or when separated) may be selected to facilitate placement of each of the electrodes 28 at the desired locations.

The substrate core 12 may be fabricated from any suitable electrically insulative material(s) that enable the electrode lead set 10 to function as described herein, such as, but not limited to, polyester (e.g., Mylar®) and/or polyimide (e.g., Kapton®). Moreover, the substrate core 12 may have any suitable thickness(es) that enables the electrode lead set 10 to function as described herein, such as, but not limited to, a thickness of between about 0.003 inches (0.0762 millimeters) and about 0.010 inches (0.254 millimeters). The substrate core 12 may have any suitable size and/or shape that enables the electrode lead set 10 to function as described herein. In the exemplary embodiment, the substrate core 12 is generally planar, but the core 12 need not be planar.

The electrically conductive pathways 30 may be fabricated from any suitable electrically conductive material(s), and may have any suitable structure, that enable the electrically conductive pathways 30 to electrically connect the electrodes 28 to the monitoring or other electronic device, such as, but not limited to, traces, coatings, layers, wires, generally planar (flat) conductors, and/or cables of silver, aluminum, gold, copper, other metallic conductors, other non-metallic conductors, electrically conductive inks, other electrically conductive coatings, and/or the like. In the exemplary embodiment, the electrically conductive pathways 30 are formed on a surface 33 of the substrate core 12 and at least partially coated with any suitable insulative material(s) (not shown), such as, but not limited to, polyvinyl chloride, polyethylene, and/or Electrodag® 1015 (commercially available from Acheson Colloids Company of Port Huron, Mich.). However, one or more of the electrically conductive pathways 30 may alternatively be embedded within the substrate core 12 or formed on a surface 35 of the substrate core 12 that is opposite the surface 33.

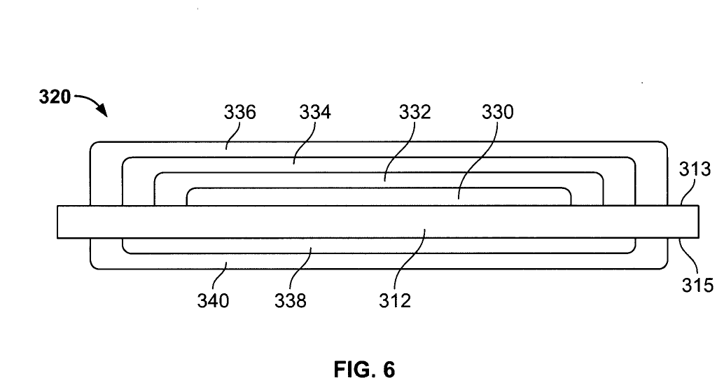

Optionally, the electrically conductive pathways 30 may be shielded using any suitable arrangement, configuration, structure, means, and/or the like, for example, but not limited to, as shown in FIG. 6. FIG. 6 is a cross-sectional view of an alternative embodiment of an electrode lead set branch 320. The branch 320 includes an electrically insulative substrate core 312 and an electrically conductive pathway 330 formed on a surface 313 of the substrate core 312. The electrically conductive pathway 330 is covered by any suitable insulative material(s) 332, such as, but not limited to, polyvinyl chloride, polyethylene, and/or Electrodag® 1015. The insulative material 332 is covered by any suitable electrically conductive material(s) 334, which is covered by any suitable insulative material(s) 336, such as, but not limited to, polyvinyl chloride, polyethylene, and/or Electrodag® 1015. A surface 315 of the substrate core 312 that is opposite the surface 313 includes any suitable electrically conductive material(s) 338 formed thereon, which is covered by any suitable insulative material(s) 340, such as, but not limited to, polyvinyl chloride, polyethylene, and/or Electrodag® 1015.

The electrodes 28 may each be any suitable type of electrode that enables the electrodes 28 to function as described herein, such as, but not limited to, known ECG electrodes and/or suitable electrodes not currently known. For example, the electrodes 28 may be, but are not limited to being, of the type that includes an electrically conductive metal or other generally solid material and/or of the type that includes an electrically conductive fluid or gel. For example, the electrodes 28 may be, but are not limited to being, conventional snap-fit electrodes, conventional tape electrodes, conventional tab electrodes, an electrically conductive pad connected to the pads 18, and/or electrodes that include an electrically conductive fluid or gel contained within a membrane. Another example of the electrodes 28 includes an electrically conductive fluid or gel applied directly to the substrate core 12 at the pads 18 (and directly or indirectly electrically connected to the corresponding electrically conductive pathway 30) and/or applied directly to an intermediate electrical contact (e.g., the intermediate electrical contact 31) embedded within or on a surface of the corresponding pad 18. The electrodes 28 may be connected to the pads 18 using any suitable structure and/or means, such as, but not limited to, using stiction, an adhesive, and/or a conventional snap-fit connection.

The electrically conductive pathways 30 may be electrically connected to the electrodes 28 using any suitable structure and/or means. In the exemplary embodiment, each electrically conductive pathway 30 is indirectly electrically connected to the corresponding electrode 28 via an intermediate electrical contact 31 that is electrically connected to the corresponding electrode 28. In the exemplary embodiment, the intermediate electrical contact 31 is an electrically conductive pad that is formed on a surface 35 of the corresponding pad 18 and is electrically connected to the corresponding electrically conductive pathway 30 through a hole (not shown) within the corresponding pad 18. Alternatively, the intermediate electrical contact 31 may be embedded within the corresponding pad 18 and electrically connected to the corresponding electrode 28 through a hole (not shown) within the corresponding pad 18 or may be on a surface 33 of the pad 18. Moreover, the intermediate electrical contact may alternatively be a conventional snap-fit contact (not shown) that extends outward from the surface 35 of the corresponding pad 18 and connects to the corresponding electrode 28 in a snap-fit connection. Optionally, the intermediate electrical contact 31 may be and/or include an electrically conductive adhesive layer, such as, but not limited to, a layer of silver epoxy, that facilitates adhering the electrode 28 to the pad 18. Alternatively, one or more of the electrically conductive pathways 30 may be directly connected to the corresponding electrode 28. When the electrically conductive pathway 30 is embedded or formed on the surface 33, the electrically conductive pathway may be directly electrically connected to the corresponding electrode 28 through a hole (not shown) within the corresponding pad 18 or within insulative material at least partially covering the pathway 30. The electrodes 28 may be sold or supplied to healthcare providers, or an intermediate party, as part of the electrode lead set 10, whether supplied or sold as attached to the pads 18. Alternatively, the electrode lead set 10 may be supplied or sold to healthcare providers, or an intermediate party, without the electrodes 28 and the healthcare provider, or the intermediary party, may supply and attach electrodes 28 to the pads 18, for example immediately prior to application of the electrodes 28 to the body. The electrodes 28 may be packaged together with the remainder of the electrode lead set 10 (whether attached to the pads 18) or alternatively the electrode lead set 10 may be packaged without including the electrodes 28 and the electrodes 28 may be provided in a separate package or provided by the healthcare provider or intermediate party.

The electrode lead set 10 may be packaged, whether including the electrodes 28, using any suitable packaging material(s), such as, but not limited to, paper and/or plastic. The paper, plastic, and/or other material(s) may be laminated and/or coated with any suitable material(s), such as, but not limited to, a metallic foil and/or a wax. The packaging material used to package the electrode lead set 10 may be sealed, for example, to facilitate preventing damage to, contamination of, and/or degradation of the any portion of the electrode lead set, for example during storage and/or shipping. The packaging material may be sealed using any suitable structure and/or means, such as, but not limited to, heat, adhesive, compression, and/or other fastening mechanisms that are capable of providing a seal. The packaging material may be hermetically sealed, for example, to facilitate preventing damage to, contamination of, and/or degradation of the any portion of the electrode lead set 10, for example, during storage and/or shipping. Moreover, in addition to the hermetic seal, the electrode lead set 10 may also be vacuum packaged. A portion(s) or an entirety of the electrode lead set 10 may be sterilized and/or disinfected prior to packaging.

In some embodiments, the electrode lead set 10 is disposable in that the electrode lead set is intended for a single use only. As used herein, the terms “disposable” and “single use” are intended to mean that a disposable, single use, electrode lead set 10 is used for one and only one patient, and thereafter discarded. For example, a disposable, single use, electrode lead set 10 may be used for one and only one procedure (e.g., an ECG measurement procedure) on one and only one patient, and thereafter discarded. Alternatively, a disposable electrode lead set 10 may be used for a plurality of procedures (e.g., a plurality of ECG measurement procedures, the plurality of procedures may be the same type of procedure or some or all of the plurality of procedures may be different procedure types) on one and only one patient, and thereafter discarded. When used for a plurality of procedures on one patient, the disposable, single use, electrode lead set 10, as a whole, is only applied to the patient one and only one time. However, some of the electrodes 28 of the disposable, single use, electrode lead set 10 may be repositioned on the one and only one patient within the range permitted by its stem 26 to accommodate different measurement locations for different procedure types and/or to obtain more accurate measurements. In other words, the electrode lead set 10 is not considered to be removed from the patient's body, as a whole, until all of the electrodes 28 are removed from the patient's body.

The electrodes 28 may be discarded along with the remainder of a disposable, single use, electrode lead set 10 after the single procedure or plurality of procedures. The material(s), size, shape, thickness(es), and/or any other properties, attributes, and/or the like of the electrode lead set 10 may be selected to facilitate providing and/or configured the electrical lead set 10 as disposable and single use. For example, material(s), size, shape, thickness(es), and/or any other properties, attributes, and/or the like of the substrate core 12, including but not limited to portions of the substrate core 12 such as the branches 20, may be selected to facilitate the providing and/or configuring the electrical lead set 10 as disposable and single use. The electrode lead set 10 may be configured and provided as disposable and single use, for example, to facilitate trying to reduce or prevent patient to patient infection and/or to facilitate trying to reduce or prevent operational costs, time, and/or workload resulting from sterilization and/or disinfection processes.

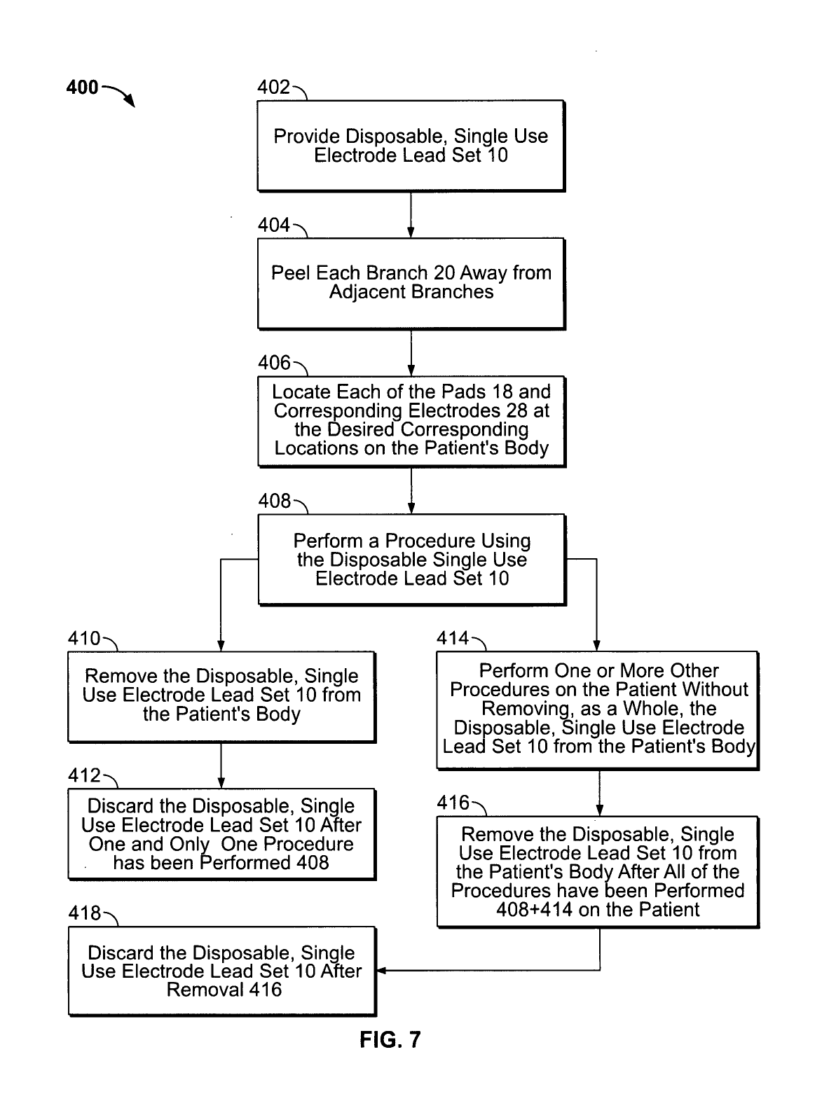

FIG. 7 illustrates one exemplary embodiment of a method 400 of using the electrode lead set embodiments described and illustrated herein. The method 400 includes providing 402 a disposable, single use, electrode lead set 10 for use with one and only one patient, peeling 404 each of the plurality of branches 20 away from adjacent branches 20, and locating 406 each of the pads 18 and the corresponding electrodes 28, independently from one another, at the desired corresponding locations on the patient's body. Each branch 20 may be peeled 404 and located 406 prior to peeling 404 some or all of the other branches 20 (e.g., the branches 20 are peeled 404 and located 406 in succession, in any order; or two or more branches 20 may be peeled 404 and located 406 prior to peeling some of the other branches 20). Alternatively, all of the branches 20 are peeled 404 before any are located 406. The method 400 also includes performing 408 a procedure on the patient using the disposable, single use, electrode lead set 10. Optionally, the method 400 may include removing 410 the disposable, single use, electrode lead set 10 from the patient's body and discarding 412 the disposable, single use, electrode lead set 10 after one and only one procedure has been performed 408 on the patient using the set 10. Removal 410 includes removing all of the electrodes 28 from the patient's body. Moreover, the method 400 may alternatively include performing 414 one or more other procedures on the patient (whether some or all of the procedures are the same or different procedure types) without removing, as a whole, the disposable, single use, electrode lead set 10 from the patient's body, removing 416 the disposable, single use, electrode lead set 10 from the patient's body after all of the procedures have been performed 408 and 414 on the patient, and discarding 418 the disposable, single use, electrode lead set 10 after the removal 416. Removal 416 includes removing all of the electrodes 28 from the patient's body. In some embodiments, one or more electrodes 28 may be repositioned on the patient's body between procedures. The method 400 is meant as exemplary only. Embodiments of disposable electrode lead sets as described and illustrated herein are not limited to the exemplary method embodiment 400.

FIG. 8 is a schematic diagram of an exemplary embodiment of an ECG system 1000 that the electrode lead set embodiments described and illustrated herein may be, but are not limited to being, used with. The ECG system 1000 includes the electrode lead set 10 and an ECG monitoring device 1002. The base portion 22 of the electrode lead set 10 is connected to the ECG monitoring device 1002 such that the electrically conductive pathways 30 of each of the electrodes 28 are electrically connected to one or more corresponding circuits (not shown) of the ECG monitoring device 1002. The electrically conductive pathways 30 may be exposed through the substrate core 12 or any insulative material covering the electrically conductive pathways 30 adjacent the base portion 22 to facilitate electrically connecting the electrically conductive pathways 30 with the ECG monitoring device 1002. The base portion 22 of the set 10 may directly connect to the ECG monitoring device 1002, or may connect to the device 1002 using any suitable extension 1006 as is shown in the exemplary embodiment. The ECG monitoring device 1002 may be any suitable processing device that is capable of performing signal processing and computational operations to convert the raw electrical signals from the electrodes 28 into meaningful ECG information that can be displayed on a monitor 1004 and/or printed out for review by a physician.

In operation, and referring to FIGS. 1 and 8, the base portion 22 of the substrate core 12 is placed on or adjacent a patient's body. The substrate core 12 is then separated into the branches 20, such as, but not limited to, one branch 20 at a time. Once separated from the adjacent branches 20, each branch 20 is manipulated to place the corresponding electrode 28 at a desired location on the patient's body. In the exemplary embodiment of FIG. 8, electrodes 28a are placed at six different locations on a chest region of the patient's body, and the electrodes 28b are placed on the arms and legs of the patient. Specifically, and for example, the electrodes 28a of six of the branches 20 are placed at the prescribed ECG precordial locations V₁, V₂, V₃, V₄, V₅, and V₆ of the AHA and the electrodes 28b of four branches 20 are placed on the prescribed limb locations RA, LA, RL, and LL of the AHA. However, the ECG system 1000 is not limited to using ten electrodes 28, is not limited to the specific locations shown, and each of the electrodes 28 is not limited to being placed at the corresponding location shown. Rather, the ECG system 1000 may use any number of electrodes 28 each located at any suitable location on the patient's body for performing ECG measurements. The particular locations shown in FIG. 8 as well as which electrode 28 of the set 10 is placed at such locations is meant as exemplary only. For example, the set 10 may include more or less branches 20 and electrodes 28 than 10, and/or the system 1000 may use more than one electrode lead set (e.g., a set for the chest region and a different set for the limbs). Different locations (e.g., different locations on the chest region and/or the limbs) than those shown may be used in addition or alternative to the locations shown. Moreover, the position of some or all of the electrodes 28 shown in FIG. 8 may be interchanged with one or more other electrodes 28 from the set 10 such that one or more electrodes 28 of the set 10 occupies a different one of the locations shown in FIG. 8 than as is shown in FIG. 8. In the exemplary embodiment, each electrode 28 is intended to be placed at only one specific location on the body (which as indicated in the directly preceding sentence may be a different location than that shown in FIG. 8). However, the locations of some or all of the electrodes 28 of the set 10 may alternatively be interchangeable.

Once all of the electrodes 28 are placed at the desired locations on the patient's body, the ECG monitoring device 1002 receives electrical signals of the electrodes 28 and converts the signals into meaningful ECG information. In some embodiments, the electrode lead set 10 is discarded after a single ECG procedure is performed on the patient or is discarded after a plurality of ECG procedures are preformed on the same patient.

In an alternative embodiment, the electrically conductive pathways 30 are electrically connected to a hand-held patient monitor 1102, as shown in FIG. 9. FIG. 9 is a schematic diagram of an exemplary embodiment of an ECG system 1100 that the electrode lead set embodiments described and illustrated herein may be, but are not limited to being, used with. The hand-held patient monitor 1102 may be any suitable processing device that is capable of performing signal processing and computational operations to convert the raw electrical signals from the electrodes 28 into meaningful ECG information that can be displayed on a monitor 1104 and/or printed out for review by a physician. In another alternative embodiment, the electrically conductive pathways 30 are electrically connected to a wireless transceiver (not shown) such that the ECG signals are transmitted to the hand-held patient monitor 1104 and/or the ECG monitoring device 1002 (FIG. 8) via a wireless connection.

The embodiments thus described provide an electrode lead set having electrically conductive pathways that may be less likely to entangle, that may accommodate differently sized and/or shaped patient bodies, and/or that may facilitate reducing patient-to-patient infection.

Although the electrode lead set embodiments are described and illustrated herein for use with an ECG system, the electrode lead set embodiments described and illustrated herein are not limited to being used with ECG systems for taking ECG measurements. Rather, the electrode lead set embodiments described and illustrated herein may be used with any system for measuring any physiologic information or performing any physiologic procedure, such as, but not limited to, for performing an electroencephalogram (EEG) procedure, for performing muscle and/or nerve stimulation and/or therapy, and/or for performing an electrophysiologic procedure. In some embodiments, the electrode lead sets described and illustrated herein may be a hybrid set that may be used to perform a plurality of different types of physiologic measurements and/or procedures.

Exemplary embodiments are described and/or illustrated herein in detail. The embodiments are not limited to the specific embodiments described herein, but rather, components and/or steps of each embodiment may be utilized independently and separately from other components and/or steps described herein. Each component, and/or each step of one embodiment, can also be used in combination with other components and/or steps of other embodiments. For example, although specific sensor elements are described and/or illustrated with specific attachment devices, each described and/or illustrated sensor element may be used with any of the described and/or illustrated attachment devices as is appropriate. When introducing elements/components/etc. described and/or illustrated herein, the articles “a”, “an”, “the”, “said”, and “at least one” are intended to mean that there are one or more of the element(s)/component(s)/etc. The terms “comprising”, “including” and “having” are intended to be inclusive and mean that there may be additional element(s)/component(s)/etc. other than the listed element(s)/component(s)/etc. Moreover, the terms “first,” “second,” and “third,” etc. in the claims are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

While the invention has been described in terms of various specific embodiments, those skilled in the art will recognize that the invention can be practiced with modification within the spirit and scope of the claims.

What is claimed is:

1. An electrode lead set for electrical connection to a body, said electrode lead set comprising:
a flexible substrate core extending between a proximal end portion and a distal end portion, the distal end portion comprising a plurality of branch end portions that are each configured to hold an electrode, the substrate core being separable into a plurality of branches that are each joined to adjacent branches by a separable interface prior to separation, each of the plurality of branches comprising a corresponding one of the branch end portions, wherein prior to separation the plurality of branches are configured such that the branch end portions are maintained in an array at the distal end portion of the substrate core; and
an electrically conductive pathway extending along each branch from the corresponding branch end portion toward the proximal end portion of the substrate core.

2. The electrode lead set according to claim 1, wherein the branch end portions each comprise a pad that is integrally formed as a portion of the substrate core, and each pad is configured to hold the corresponding electrode.

3. The electrode lead set according to claim 1, wherein the substrate core comprises a base portion that includes the proximal end portion, each branch comprises a stem extending from the base portion to the corresponding branch end portion, and each electrically conductive pathway extends from the corresponding branch end portion along the stem and at least a portion of the base portion.

4. The electrode lead set according to claim 1, wherein the array comprises a pair of branch end portions arranged on opposite sides of a longitudinal axis of the substrate core prior to separation of the branches.

5. The electrode lead set according to claim 4, wherein each branch end portion of the pair of branch end portions is located at about the same distance from the proximal end portion of the substrate core prior to separation of the branches.

6. The electrode lead set according to claim 1, wherein the array comprises a first pair of branch end portions arranged on opposite sides of a longitudinal axis of the substrate core, the array comprises a second pair of branch end portions arranged on opposite sides of the longitudinal axis, each of the first pair of branch end portions is located at about the same first distance from the proximal end portion of the substrate core prior to separation of the branches, and each of the second pair of branch portions is located at about the same second distance from the proximal end portion that is different than the first distance prior to separation of the branches.

7. The electrode lead set according to claim 1, wherein the array of branch end portions comprises branch end portions arranged in a uniform pattern on opposite sides of a longitudinal axis of the substrate core.

8. The electrode lead set according to claim 7, wherein the uniform pattern is symmetrical about the longitudinal axis.

9. The electrode lead set according to claim 1, wherein the array comprises a tiered configuration.

10. The electrode lead set according to claim 1, wherein the array of branch end portions comprises a first branch end portion that is located a greater distance from a central longitudinal axis of the substrate core than the second branch end portion.

11. The electrode lead set according to claim 1, wherein the electrode lead set is a disposable, single use, electrode lead set.

12. The electrode lead set according to claim 1, wherein the substrate core comprises one of polyester and polyimide.

13. The electrode lead set according to claim 1, wherein the substrate core comprises a base portion that includes the proximal end portion, and, after separation of the branches, each branch is only connected to adjacent branches at the base portion.

14. The electrode lead set according to claim 1, wherein after separation of the branches, each branch end portion is selectively positionable at a plurality of different positions relative to the branch end portion of each of the other branches such that each branch end portion can be positioned at about the same location on at least one of differently sized and differently shaped bodies.

15. The electrode lead set according to claim 1, wherein the electrically conductive pathways are shielded.

16. The electrode lead set according to claim 1, wherein the array of branch end portions comprises a first branch end portion located at a greater distance from the proximal end portion of the substrate core than a second branch end portion.

17. An electrode lead set assembly for electrical connection to a body, said electrode lead set assembly comprising:
a flexible substrate core extending between a proximal end portion and a distal end portion, the distal end portion comprising a plurality of branch end portions, the substrate core being separable into a plurality of branches that are each joined to adjacent branches by a separable interface prior to separation, each of the plurality of branches comprising a corresponding one of the branch end portions, wherein prior to separation the plurality of branches are configured such that the branch end portions are maintained in an array at the distal end portion of the substrate core; and
a plurality of electrodes each being held by a corresponding branch end portion of a different branch of the plurality of branches; and
a plurality of electrically conductive pathways, each electrically conductive pathway extending along a different branch of the plurality of branches, each electrically conductive pathway being electrically connected to the corresponding electrode held by the corresponding branch, wherein each electrically-conductive pathway extends from the corresponding branch end portion toward the proximal end portion of the substrate core.

18. The electrode lead set assembly according to claim 17, wherein the branch end portions each comprise a pad that is integrally formed as a portion of the substrate core, and each pad holds the corresponding electrode.

19. The electrode lead set assembly according to claim 17, wherein the substrate core comprises a base portion that includes the proximal end portion, each branch comprises a stem extending from the base portion to the corresponding branch end portion, and each electrically conductive pathway extends from the corresponding branch end portion along the stem and at least a portion of the base portion.

20. The electrode lead set assembly according to claim 17, wherein the array comprises a first pair of branch end portions arranged on opposite sides of a longitudinal axis of the substrate core, the array comprises a second pair of branch end portions arranged on opposite sides of the longitudinal axis, each of the first pair of branch end portions is located at about the same first distance from the proximal end portion of the substrate core prior to separation of the branches, and each of the second pair of branch portions is located at about the same second distance from the proximal end portion that is different than the first distance prior to separation of the branches.

21. The electrode lead set assembly according to claim 17, wherein the array comprises a tiered configuration.

22. The electrode lead set assembly according to claim 17, wherein the electrode lead set is a disposable, single use, electrode lead set.

23. The electrode lead set assembly according to claim 17, wherein the substrate core comprises at least one of polyester and polyimide.

24. The electrode lead set assembly according to claim 17, wherein each of the electrically conductive pathways is electrically connected to a monitoring device.

25. The electrode lead set assembly according to claim 24, wherein the monitoring device comprises a hand-held patient monitor.
